(19) United States
(12) Patent Application Publication
(10) Pub. No.: US 2025/0152306 A1
(43) Pub. Date: May 15, 2025

(54) SYSTEMS AND METHODS FOR TREATMENT PLAN COMPARISON

(71) Applicant: Align Technology, Inc., San Jose, CA (US)

(72) Inventors: Mitra Derakhshan, Herndon, VA (US); Angelo Maura, Amsterdam (NL)

(21) Appl. No.: 19/025,585

(22) Filed: Jan. 16, 2025

Related U.S. Application Data
(63) Continuation of application No. 18/504,071, filed on Nov. 7, 2023.
Application No. 18/504,071 is a division of application No. 14/695,921, filed on Apr. 24, 2015, now Pat. No. 11,850,111.

(51) Int. Cl. A61C 7/00; A61C 7/08; G16H 50/50

(52) U.S. Cl. CPC A61C 7/002; A61C 7/08; G16H 50/50

(57) ABSTRACT

Methods for planning an orthodontic treatment are provided, including: transmitting an initial tooth data set to an orthodontic treatment planning tool, the initial tooth data set including an initial arrangement of a patient's teeth; receiving a plurality of treatment plans for the patient's teeth from the orthodontic treatment planning tool, where each treatment plan includes a plurality of treatment stages to move the patient's teeth from the initial arrangement toward a respective final arrangement, and where the plurality of treatment plans differ from each other based on one or more treatment factors; receiving, through a display, a comparison of a treatment stage of a first treatment plan of the plurality of treatment plans to a treatment stage of a second treatment plan of the plurality of treatment plans; and providing a user input selecting one of the first treatment plan or the second treatment plan based on the comparison.

### CROSS-REFERENCE TO RELATED APPLICATIONS

This application is a continuation of U.S. patent application Ser. No. 18/504,071, filed Nov. 7, 2023, which is a divisional of U.S. patent application Ser. No. 14/695,921, filed Apr. 24, 2015, now U.S. Pat. No. 11,850,111, issued Dec. 26, 2023, the contents of which are included by reference in its entirety.

### BACKGROUND OF THE INVENTION

The present invention relates generally to the field of orthodontics. More particularly, the invention relates to systems and methods of comparative orthodontic treatment planning.

A primary objective of orthodontics is to realign patients' teeth to positions where the teeth function optimally and have an aesthetically pleasing appearance. The goal of a doctor is to take the patient from his or her current condition (“initial arrangement”) to a treatment goal (“final arrangement”). There may be many different ways to achieve the goal and these are known as “treatment options.” The methodologies used by the doctor to help the patient reach the goal are known as “treatment plans.”

Typically, appliances such as fixed braces and wires are applied to a patient's teeth to gradually reposition them from an initial arrangement to a final arrangement. As noted above, braces (wires and brackets) have been used for several decades to straighten and maintain tooth positioning. Over time and with a series of clinical visits and adjustments to the braces, the orthodontist adjusts the wires and brackets to move the teeth to their final position. However, as wires and brackets can be unsightly, uncomfortable, and cumbersome for the patient, removable polymeric shell appliances (e.g., aligners) were developed to straighten teeth. Clear aligners, such as the Invisalign® aligners manufactured by Align Technology, Inc. of San Jose, Calif., are virtually invisible as well as more convenient. Unlike patients with braces, the patient wearing aligners has the freedom to eat anything he or she desires because the aligners are removable. Furthermore, removable aligners allow for easier care and cleaning of teeth.

The Invisalign® System includes designing and/or fabricating multiple, and usually all, of the aligners to be worn by the patient before the aligners are administered to the patient and used to reposition the teeth (i.e., at the outset of treatment). Often, designing and planning a customized treatment for a patient includes using computer-based three-dimensional planning and design tools, such as ClinCheck® from Align Technology, Inc. The design of the aligners can rely on computer modeling of a series of planned successive tooth arrangements, and the individual aligners are designed to be worn over the teeth and elastically reposition the teeth to each of the planned tooth arrangements.

Typically, the treatment plan is based on one prescription form, which is based on one input and provides one output. However, different clinicians may vary in their definitions of individual orthodontic parameters and their view of how a case should ideally be treated will also often vary. Patients may also have different goals as well as different budgets. As a set of aligners is typically made prior to starting treatment, there is a need for a robust comparative treatment planning tool that allows both patients and doctors to view different options when making treatment planning decisions. In view of the foregoing, it would be desirable to have methods and systems to provide an interactive and dynamic orthodontic treatment planning tool.

### SUMMARY OF THE INVENTION

In accordance with an embodiment, a method is provided for orthodontic treatment planning with a comparative orthodontic treatment planning tool. Initial tooth data set comprising an initial tooth arrangement is received at the treatment planning tool. The initial tooth data set is used to generate at least one treatment plan to move teeth from the initial arrangement. Each treatment plan comprises a plurality of treatment stages. A user is allowed to compare tooth positioning of at least two different treatment stages by displaying the at least two different treatment stages on a screen. The user is then allowed to select an end stage based on comparison of tooth positioning of at least two different stages.

In accordance with another embodiment, a system is provided for comparative orthodontic treatment planning. The system includes a computer coupled to a server, and the computer comprises a processor and a computer readable medium comprising instructions which, if executed, causes the computer to generate at least one treatment plan for a patient based on an initial tooth data set received by the computer. The plan comprises a plurality of planned successive tooth arrangements for moving teeth along a treatment path from an initial arrangement toward a selected final arrangement. The plan also comprises a series of one or more treatment stages to move teeth along the treatment path. The computer readable medium comprising instructions which, if executed, also causes the computer to display tooth arrangements for each of the one or more treatment stages, to allow a user to compare tooth arrangements for at least two different treatment stages, and to allow a user to select a treatment plan after comparing tooth arrangements.

In accordance with yet another embodiment, a method is provided for orthodontic treatment planning using an orthodontic treatment planning system comprising a computer and a comparative treatment planning tool. At the computer, an initial tooth data set comprising an initial tooth arrangement is received. The comparative treatment planning tool and the initial tooth data set are used to generate a plurality of different treatment plans to move teeth from the initial arrangement toward a target arrangement. Each of the treatment plans comprises a plurality of treatment stages. A user is allowed to select a desired treatment plan based on tooth positioning of target arrangements of at least two different treatment plans by simultaneously displaying on a screen the target arrangements of each of at least two different treatment plans.

### BRIEF DESCRIPTION OF THE DRAWINGS

The invention, together with further objects and advantages thereof, may best be understood by reference to the following description taken in conjunction with the accompanying drawings in which:

FIG. 1 is a perspective view of a dental appliance in accordance with an embodiment.

FIG. 2 is a flow chart of a process for treatment planning and fabrication of orthodontic appliances in accordance with an embodiment.

FIG. 3A is a screenshot of a comparative treatment planning tool illustrating a graphical representation of a treatment stage in a treatment plan in accordance with an embodiment.

FIG. 3B is a screenshot of a comparative treatment planning tool illustrating a graphical representation of a split screen screenshot comparing different treatment stages in accordance with an embodiment

FIG. 3C is a screenshot of a comparative treatment planning tool illustrating a graphical representation of a treatment stage superimposed over another treatment stage in accordance with an embodiment.

FIG. 4 is a screenshot of a comparative treatment planning tool illustrating a graphical representation of a treatment stage in a treatment plan in accordance with a different embodiment.

FIG. 5 is a screenshot of a comparative treatment planning tool illustrating a graphical representation of a treatment stage in a treatment plan in accordance with another embodiment.

FIG. 6 is a screenshot of a comparative treatment planning tool illustrating a graphical representation of a treatment stage in a treatment plan in accordance with yet another embodiment.

FIG. 7 is a flow chart illustrating a method of orthodontic treatment planning with a comparative orthodontic treatment planning tool in accordance with an embodiment.

FIG. 8 is a flow chart illustrating a method of orthodontic treatment planning using an orthodontic treatment planning system comprising a computer and a comparative treatment planning tool in accordance with an embodiment.

### DETAILED DESCRIPTION OF THE PREFERRED EMBODIMENTS

The present invention provides improved systems and methods of managing delivery of an orthodontic treatment plan that can be customized for the individual patient being treated. The systems and methods of the present invention include generating one or more treatment plans for a patient and providing additional options for customizing the treatment plans. A comparative orthodontic treatment planning tool provides dentists and orthodontists with the ability to collaborate with patients and fabricators to make informed decisions in orthodontic treatment planning Comparative treatment planning tools described herein provide patients with more opportunity for customization and budget planning, and collaborative decision making with the orthodontist. Orthodontist can also use the comparative treatment planning tools to work with specialists, such as oral surgeons, prosthodontists, and restorative specialists, and generalists can use the comparative treatment planning tools to work with orthodontists and other specialists.

In various embodiments, a comparative treatment planning tool that is used with a computing system can be used in orthodontic treatment planning and the design of dental appliances thereof. Such a computing system may include one or more computing devices having, for example, a processor and memory. The memory can include various types of information including data (e.g., dentition measurement, uploaded scanned tooth data, and/or digital model data) and/or executable instructions to perform the methods discussed herein. Various embodiments can include one or more input and/or output interfaces. Such interfaces can be used, for instance, to connect the computing device with one or more input or output devices. For example, a system may include connectivity to a scanning device, a camera dock, a keyboard, and/or other peripherals.

Some embodiments can include a network interface. Such an interface can allow, for example, for processing on another networked computing device. Such devices can be used to obtain information about the patient or executable instructions for use with various embodiments provided herein, in some instances.

The comparative treatment planning tools described herein provide doctors, patients, and manufacturers with more information as well as additional options in treatment planning, product selection and the ordering process. The comparative treatment planning tools also serve to inform and educate patients with multiple different treatment options (and their respective pricing) along with visual tools that can show predicted teeth arrangements at different stages for different options. The treatment plan can be customized to the individual patient and typically account for patient-specific factors, such as the positioning of the patient's teeth, desired movements, treatment goals and timing, pricing, complexity of treatment, and other clinical considerations.

In addition, the comparative treatment planning tools described herein can improve shipping to doctor, reduction in number of aligners, progress tracking to ensure better treatment outcomes, or material selection for better outcomes. For example, if an orthodontist or dentist decides on partial treatment planning in steps or decides to use different materials or additional or different features for a particular stage, then aligners can be batch shipped for a particular stage of the treatment plan. Thus, the comparative treatment planning tools described herein provide additional flexibility to treatment planners in that treatment planning can be done all at once from beginning to end or treatment planning can be done in particular stages. Even if treatment planning is done in stages, the comparative treatment planning tool still provides an overview of the treatment plan from beginning to end.

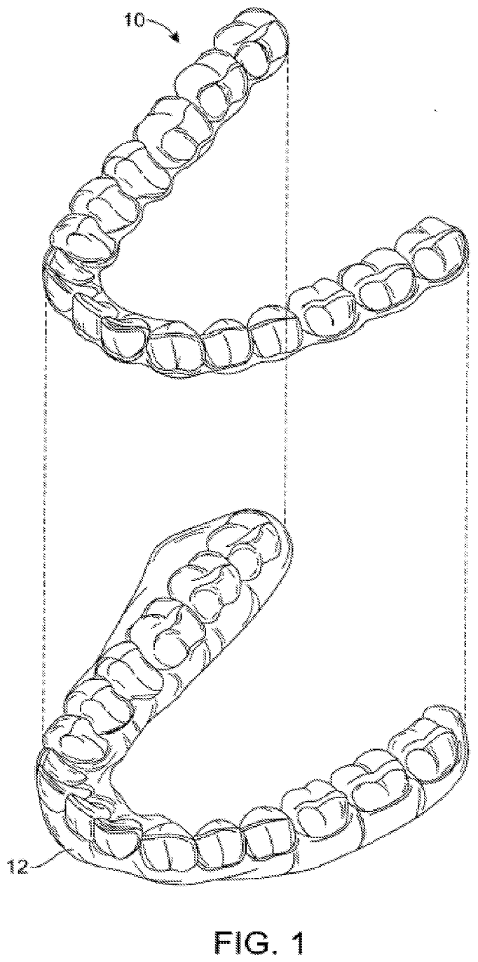

FIG. 1 shows an aligner or adjustment appliance 10, which is worn by the patient in order to achieve an incremental repositioning of individual teeth in the jaw 12 as described generally above. The appliance can include a shell (e.g., polymeric shell) having teeth-receiving cavities that receive and resiliently reposition the teeth. Such appliances, including those utilized in the Invisalign® System, are described in numerous patents and patent applications assigned to Align Technology, Inc. including, for example in U.S. Pat. Nos. 6,450,807, and 5,975,893, as well as on the company's website, which is accessible on the World Wide Web (see, e.g., www.align.com). As shown in FIG. 1, the appliance or aligner 10 has a geometry for receiving teeth.

As noted above, aligners have been developed as a more aesthetically pleasing as well as more convenient appliance for straightening teeth. An aligner is a relatively, and typically transparent, thin shell of material that generally conforms to a patient's teeth but is slightly out of alignment with the current (i.e., initial) tooth configuration. The material of the aligner is typically a polymeric material that has resilient properties. Dental treatments using aligners typically involve repositioning misaligned teeth and changing bite configurations for improved cosmetic appearance and dental function. Such repositioning with aligners can be accomplished by applying controlled forces to one or more teeth over a period of time.

As shown in FIG. 1, the appliance 10 can be configured to fit over an entire dental arch 12. In other embodiments, the appliance may be designed to fit over some or all of the teeth in the upper or lower jaw. In some embodiments, the appliance is a removable orthodontic tooth positioning appliance having teeth receiving cavities shaped to directly receive at least some of the patient's teeth and apply a resilient positioning force to the patient's teeth. In some cases, only certain teeth received by an appliance will be repositioned by the appliance while other teeth can provide a base or anchor region for holding the appliance in place as it applies force against the tooth or teeth targeted for repositioning. In some cases, many or most, and even all, of the teeth will be repositioned at some point during treatment.

Teeth that are moved can also serve as a base or anchor for holding the appliance as it is worn by the patient. Typically, no wires or other means will be provided for holding an appliance in place over the teeth. In some cases, however, it may be desirable or necessary to provide individual anchors on teeth with corresponding receptacles or apertures in the appliance so that the appliance can apply a selected force on the tooth. Exemplary appliances are described in numerous patents and patent applications assigned to Align Technology, Inc. including, for example, in U.S. Pat. Nos. 6,450,807, and 5,975,893, as well as on the company's website, which is accessible on the World Wide Web (see, e.g., www.invisalign.com). The aforementioned patents are hereby incorporated herein for all purposes.

An appliance can be designed and/or provided as part of a set of a plurality of appliances. In such an embodiment, each appliance may be configured such that a tooth-receiving cavity has a geometry corresponding to an intended intermediate or final tooth arrangement. The patient's teeth can be progressively repositioned from an initial tooth arrangement to a target tooth arrangement by placing a series of incremental position adjustment appliances over the patient's teeth. A target tooth arrangement is a planned tooth arrangement (e.g., a planned temporary or final arrangement) selected for the patient's teeth at a specific stage of the planned orthodontic treatment. A target arrangement can be one of many intermediate arrangements for the patient's teeth during the course of orthodontic treatment, which may include where surgery is recommended, where inter-proximal reduction (IPR) is appropriate, where extraction is appropriate, where a progress check is scheduled, where anchor placement is best, where palatal expansion is desirable, etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

Each appliance may be configured so that its tooth-receiving cavity has a geometry corresponding to an intermediate or final tooth arrangement intended for the appliance. The patient's teeth are progressively repositioned from their initial tooth arrangement to a final tooth arrangement by placing a series of incremental position adjustment appliances over the patient's teeth. The adjustment appliances can be generated all at the same stage or in sets or batches, e.g., at the beginning of a stage of the treatment. A set of different appliances can be designed and fabricated prior to the patient wearing any appliance in the set. The patient wears each appliance for a set period of time or until the pressure of each appliance on the teeth is minimal or can no longer be felt. At that point, the patient replaces the current adjustment appliance with the next adjustment appliance in the series until no more appliances remain. The appliances are generally not affixed to the teeth so as to require removal by a practitioner (e.g., patient removable) and the patient may place and replace the appliances at any time during the procedure. The final appliance or several appliances in the series may have a geometry or geometries selected to overcorrect the tooth arrangement, i.e., have a geometry which would (if fully achieved) move individual teeth beyond the tooth arrangement which has been selected as the “final.” Such over-correction may be desirable in order to offset potential relapse after the repositioning method has been terminated, i.e., to permit movement of individual teeth back toward their pre-corrected positions. Over-correction may also be beneficial to increase the rate of correction, i.e., by having an appliance with a geometry that is positioned beyond a desired intermediate or final position, the individual teeth will be shifted toward the position at a greater rate. In such cases, the use of an appliance can be terminated before the teeth reach the positions defined by the appliance.

Individual aspects of a treatment planning process are discussed in further detail below. The process includes generating a treatment plan for repositioning a patient's teeth. Briefly, a treatment plan will include obtaining data comprising an initial arrangement of the patient's teeth, which typically includes obtaining an impression or scan of the patient's teeth prior to the onset or start of treatment. The treatment plan will also include identifying a desired final arrangement of the patient's teeth, as well as a plurality of planned successive or intermediary tooth arrangements for moving the teeth along a treatment path from the initial arrangement toward the selected final arrangement. As noted, treatment can be pre-planned for administration to a patient in a series of one or more treatment phases, with a phase including a set of appliances that are worn successively by the patient to reposition the teeth through planned arrangements and eventually toward a selected final arrangement.

The adjustment appliances can be generated all at the same stage or in sets or batches, e.g., at the beginning of a stage of the treatment, and the patient wears each appliance until the pressure of each appliance on the teeth can no longer be felt or has resulted in the maximum amount of expressed tooth movement for that given stage. A plurality of different appliances (e.g., set) can be designed and even fabricated prior to the patient wearing any appliance of the appliances. After wearing an appliance for an appropriate period of time, the patient replaces the current appliance with the next appliance in the series until no more appliances remain. The appliances are generally not affixed to the teeth and the patient may place and replace the appliances at any time during the procedure (e.g., patient-removable appliances).

The final appliance or several appliances in the series may have a geometry or geometries selected to overcorrect the tooth arrangement, i.e., have a geometry which would (if fully achieved) move individual teeth beyond the tooth arrangement which has been selected as the “final.” Such over-correction may be desirable in order to offset potential relapse after the repositioning method has been terminated, i.e., to permit movement of individual teeth back toward their pre-corrected positions. Over-correction may also be beneficial to speed the rate of correction, i.e., by having an appliance with a geometry that is positioned beyond a desired intermediate or final position, the individual teeth will be shifted toward the position at a greater rate. In such cases, the use of an appliance can be terminated before the teeth reach the positions defined by the appliance.

The appliance 10 can be fabricated using a variety of different suitable methods, including thermoforming, casting, 3D printing, stereolithography, milling, direct fabrication etc. For example, methods for making the appliances can include thermoforming a polymer sheet into an aligner by heating the sheet and then molding the sheet to a particular configuration. Exemplary methods for fabricating the appliances are described in numerous patents and patent applications assigned to Align Technology, Inc. including, for example, in U.S. application Ser. No. 13/186,374 as well as on the company's website, which is accessible on the World Wide Web (see, e.g., www.invisalign.com). The aforementioned patent application is hereby incorporated herein for all purposes.

As noted above, placement of an appliance over the teeth provides controlled forces in specific locations to gradually move the teeth into a new configuration and repetition of this process with successive appliances that provide progressive configurations eventually move the teeth through a series of intermediate arrangements to a target, prescribed, or desired arrangement. An example of such a system is described in U.S. Pat. No. 5,975,893, which is hereby incorporated herein by reference. Appliances can be generated based on the planned arrangements and will be provided to the practitioner and ultimately administered to the patient. The appliances are typically provided and/or administered in sets or batches of appliances, such as 2, 3, 4, 5, 6, 7, 8, 9, or more appliances, but are not limited to any particular administrative scheme.

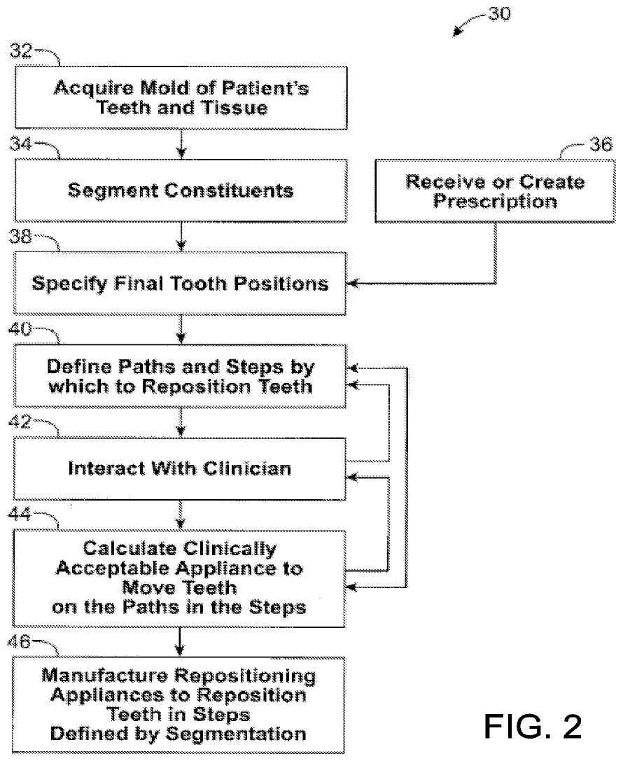

FIG. 2 illustrates the general flow of an exemplary process 30 for defining and generating a treatment plan, including repositioning appliances for orthodontic treatment of a patient. The steps of the process can be implemented as computer program modules for execution on one or more computer systems. As an initial step, an initial digital data set of the patient's teeth is created. To create the initial digital data set, a mold or a scan of a patient's teeth or mouth tissue is acquired (Step 32). Creating the initial digital data set generally involves taking casts of the patient's teeth and gums, and may in addition or alternately involve taking wax bites, direct contact scanning, x-ray imaging, tomographic imaging, sonographic imaging, and other techniques for obtaining information about the position and structure of the teeth, jaws, gums and other orthodontically relevant tissue. From the data so obtained, a digital data set is derived that represents an initial (e.g., pretreatment) arrangement of the patient's teeth and other tissues.

The initial digital data set, which may include both raw data from scanning operations and data representing surface models derived from the raw data, is processed to segment the tissue constituents from each other (Step 34), including defining discrete dental objects. For example, data structures that digitally represent individual tooth crowns can be produced. In some embodiments, digital models of entire teeth are produced, including measured or extrapolated hidden surfaces and root structures.

The desired final position of the teeth that is, the desired and intended end result of orthodontic treatment, can be calculated from basic orthodontic principles, or can be extrapolated computationally from a clinical prescription (Step 36). With a specification of the desired final positions of the teeth and a digital representation of the teeth themselves, the final position and surface geometry of each tooth can be specified (Step 38) to form a complete model of the teeth at the desired end of treatment. Generally, in this step, the position of every tooth is specified. The result of this step is a set of digital data structures that represents a desired and/or orthodontically correct repositioning of the modeled teeth relative to presumed-stable tissue. The teeth and tissue are both represented as digital data.

Having both a beginning position and a final position for each tooth, the process next defines a treatment path or tooth path for the motion of each tooth (Step 40). This includes defining a plurality of planned successive tooth arrangements for moving teeth along a treatment path from an initial arrangement to a selected final arrangement. In one embodiment, the tooth paths are optimized in the aggregate so that the teeth are moved in the most efficient and clinically acceptable fashion with the least amount of round-tripping to bring the teeth from their initial positions to their desired final positions. Round-tripping is any motion of a tooth in any direction other than directly toward the desired final position. Round-tripping is sometimes necessary to allow teeth to move past each other. The tooth paths are segmented. The segments are calculated so that each tooth's motion within a segment stays within threshold limits of linear and rotational translation. In this way, the end points of each path segment can constitute a clinically viable repositioning, and the aggregate of segment end points constitute a clinically viable sequence of tooth positions, so that moving from one point to the next in the sequence does not result in a collision of teeth. [In one embodiment, the comparative treatment planning tool allows the user to choose whether the tooth paths include round-tripping, and if so, how much round-tripping. It may be possible to stop treatment before any (significant) round tripping is necessary. In some cases, the orthodontist or dentist can provide for IPR or extraction in the treatment plan instead. In this example, the comparative treatment planning tool can also allow the orthodontist or dentist to see a comparison of round-tripping versus IPR or extraction, and then select the better option after seeing the comparison. Thus, it will be understood that there may be different treatment paths for the same initial and final position of a tooth.

The threshold limits of linear and rotational translation are initialized, in one implementation, with default values based on the nature of the appliance to be used. More individually tailored limit values can be calculated using patient-specific data. The limit values can also be updated based on the result of an appliance-calculation, which may determine that at one or more points along one or more tooth paths, the forces that can be generated by the appliance on the then-existing configuration of teeth and tissue is incapable of affecting the repositioning that is represented by one or more tooth path segments. With this information, paths or the affected subpaths can be recalculated.

At various stages of the process, the process can include interaction with a clinician responsible for the treatment and/or the patient (Step 42). The interaction can be implemented using a comparative treatment planning tool, as described herein, programmed to receive tooth positions and models, as well as path information. The comparative treatment planning tool is advantageously programmed to allow the clinician to display multiple different animations of the positions and paths and to allow the clinician to reset the final positions of one or more of the teeth and to specify constraints to be applied to the segmented paths. If the clinician makes any changes, certain steps of the process are performed again to recalculate paths.

The tooth paths and associated tooth position data are used to calculate clinically acceptable appliance configurations (or successive changes in appliance configurations) that will move the teeth on the defined treatment path in the steps specified (Step 44). Each appliance configuration corresponds to a planned successive arrangement of the teeth, and represents a step along the treatment path for the patient. The steps are defined and calculated so that each discrete position can follow by straight-line tooth movement or simple rotation from the tooth positions achieved by the preceding discrete step and so that the amount of repositioning required at each step involves an orthodontically optimal amount of force on the patient's dentition. As with other steps, this calculation step can include interactions with the clinician and/or patient (Step 42).

Having calculated appliance definitions, the process 30 can proceed to the manufacturing step (Step 46) in which appliances defined by the process are manufactured, or electronic or printed information is produced that can be used by a manual or automated process to define appliance configurations or changes to appliance configurations. Appliances according to the treatment plan can be produced in entirety, such that each of the appliances is manufactured (e.g., prior to treatment), or can be manufactured in sets or batches. For example, in some cases it might be appropriate to manufacture an initial set of appliances at the outset of treatment with the intention of manufacturing additional sets of appliances (e.g., second, third, fourth, etc.) after treatment has begun (e.g., as discussed further herein). For example, a first set of appliances can be manufactured and administered to a patient. Following administration, it may be desirable to inspect the progression of the patient's teeth along the treatment path before manufacturing and/or administering subsequent set(s) of appliances. In some cases, it may be desirable to modify the treatment plan before manufacturing subsequent set(s) of appliances.

Generating and/or analyzing digital treatment plans, as discussed herein, can include, for example, use of three-dimensional orthodontic treatment planning tools, such as ClinCheck® from Align Technology, Inc. Such treatment planning tools allow the clinician to use the actual patient's dentition as a starting point for customizing a treatment plan. The ClinCheck® technology uses a patient-specific digital model to plot a treatment plan, and then uses a processed (e.g., segmented) scan of the achieved treatment outcome, as discussed in U.S. Pat. Nos. 7,156,661 and 7,077,647, which are hereby incorporated herein for all purposes.

The design of the aligners can rely on computer modeling of a series of planned successive tooth arrangements, and the individual aligners are designed to be worn over the teeth and elastically reposition the teeth to each of the planned tooth arrangements.

The theory of orthodontic treatment is not universally agreed upon, and actual treatment and outcomes are subject to additional uncertainties of measurement of patient variables, of relationships to unmeasured patient variables, as well as of varying patient compliance and different patient goals. As a result, different clinicians might prefer different treatment plans for a single patient. Thus, a single treatment plan may not be accepted by every clinician since there is no universally accepted “correct” treatment plan. Additionally, some patients may prefer a less complex treatment plan, less expensive option, or one with a shorter treatment duration. For example, a treatment plan may require surgery in addition to the use of a set of aligners and an alternative treatment plan might involve only treatment with aligners. Some patients may opt not to have surgery and use only aligners to achieve a goal that is close enough to the ideal treatment outcome. In another example, a patient might have a date (e.g., a wedding or graduation) by which the patient would like to have his or her teeth straightened. In such a case, the patient might choose a treatment plan that has a shorter duration but still an acceptable result.

Embodiments of the treatment planning tool described herein allow the clinician to obtain a single initial data set of the patient's initial tooth arrangement and provide the clinician with different options for treatment planning to give both the clinician and patient more flexibility and treatment options. The treatment planning tool also serves to educate the patient with a visual tool to view different treatment options, based on factors, such as the complexity of treatment, pricing of the treatment, duration of the treatment, material selection, attachment selection, etc.

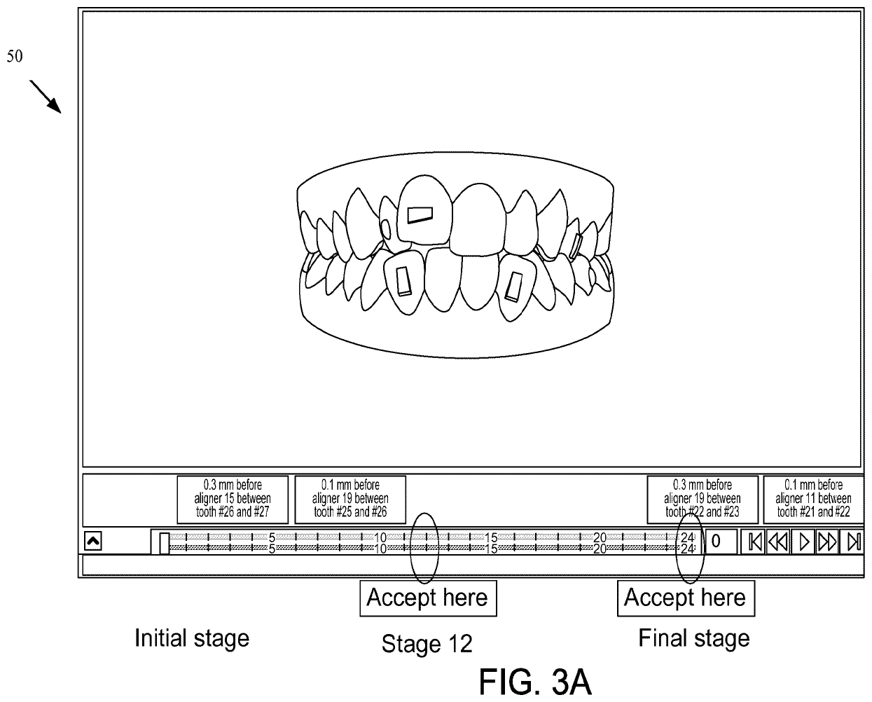

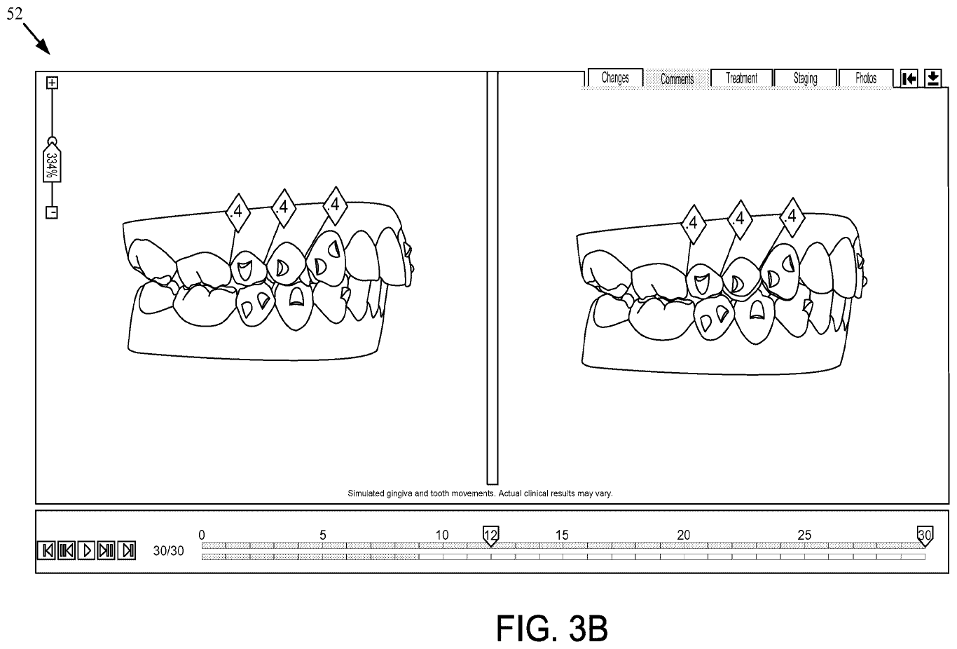

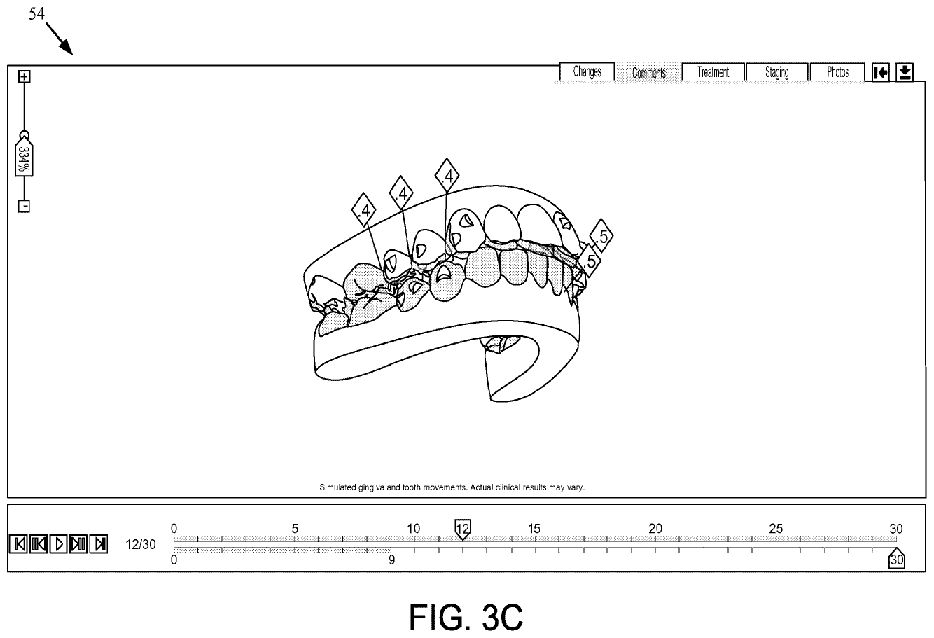

Referring to FIGS. 3-6, embodiments of a comparative orthodontic treatment planning tool for designing a series of polymeric shell appliances will be described. FIG. 3A shows a screen shot 50 of a comparative treatment planning tool illustrating a graphical representation of a treatment stage in a treatment plan according to an embodiment. In the example shown in FIG. 3A, the comparative treatment planning tool shows the predicted tooth positioning of the treatment plan at “Stage 12.” The treatment planning tool allows the user (i.e., clinician and/or patient) to see the predicted tooth positioning for a particular stage of the treatment based on the initial data set obtained. For example, the screenshot shown in FIG. 3A shows the predicted tooth positioning at “Stage 12” of a 24 stage treatment plan. In the illustrated example, the treatment planning tool allows the user to use the scroll bar at the bottom of the screen to toggle between stages to view the predicted tooth positioning at each stage. In this example, the user can then select between a 12 stage treatment plan and a 24 stage treatment plan. The 12 stage treatment plan typically costs less for the patient than the 24 stage treatment plan, and also likely has a shorter treatment length. The cost and time savings for the doctor, lab fee, and even the patient could be significant as compared to the 24 stage treatment plan. However, the tooth positioning achieved in the 12 stage treatment plan may not be as ideal as the positioning that can be achieved by the 24 stage treatment plan. According to an embodiment, the comparative treatment planning tool allows the user to toggle between the initial stage (Stage 0) and Stage 12, between the initial stage and the final stage (Stage 24), and between Stage 12 and the final stage.

In another embodiment, as shown in FIG. 3B, the comparative treatment planning tool can also provide a split screen to allow a dentist or orthodontist to compare different stages from the same treatment plan or from different treatment plans. In the exemplary screenshot 52 shown in FIG. 3B, a split screen shows the predicted positions of the teeth at Stage 12 on the left side and the predicted positions of the teeth at the final stage (Stage 30) on the right side. In the illustrated embodiment, the two stages shown in the split screen are from the same treatment plan. However, it will be understood that the comparative treatment planning tool can provide a split screen to compare different stages from different treatment plans. The split screen allows a side-by-side comparison of different stages (either from the same treatment plan or from different treatment plans).

In another embodiment, the comparative treatment planning tool can superimpose the predicted tooth positioning of one stage over the predicted tooth positioning of another stage to allow the user to see the difference between the stages (either from the same treatment plan or from different treatment plans). As shown in the screenshot 54 of FIG. 3C, the teeth can be shaded or colored differently for each stage and the different stages can be superimposed over one another so that the patient and/or clinician can see the difference of the tooth positioning between the stages.

As noted above, toggling, superimposing, and split screens can also provide comparisons of stages from different treatment plans. For example, one treatment plan may include IPR or extraction and another treatment plan does not have IPR or extraction, and the comparative treatment planning tool can be used to compare the final (or an intermediate stage) stages of the two treatment plans. In some cases, the difference between Stage 24 may not be significant enough for a patient and/or clinician to spend the additional time and/or money for the patient's teeth to reach the predicted final stage. If the patient and/or clinician decide the difference is not worth the additional expense and time, then the user can click the “Accept here” button for Stage 12, as shown in FIG. 3A. If, after comparing Stage 12 and Stage 24, the patient and/or clinician decide that it is worth the additional expense and time for the patient's teeth to reach the predicted final stage, the user can click the “Accept here” button for Stage 24, as shown in FIG. 3A. It will be understood that, in this embodiment, the tooth movement routes are the same, whether the user chooses to stop at Stage 12 or Stage 24.

According to an embodiment, the comparative treatment planning tool also allows the clinician to view multiple comparative treatment plans with restorative treatment in addition to orthodontic treatment. For example, clinician may be able to see that, at Stage 12, the teeth are not in the ideal positions with only orthodontic treatment. The comparative treatment planning tool also allows the clinician to add restorative treatment to the plan and the treatment planning tool can provide a comparison at a stage (e.g., Stage 12) of the teeth with and without the restorative treatment. Thus, if the clinician and/or patient can decide whether the restorative treatment is worth it. The comparative treatment planning tool helps clinicians and patients make better choices by providing different options and allowing comparison of the different options.

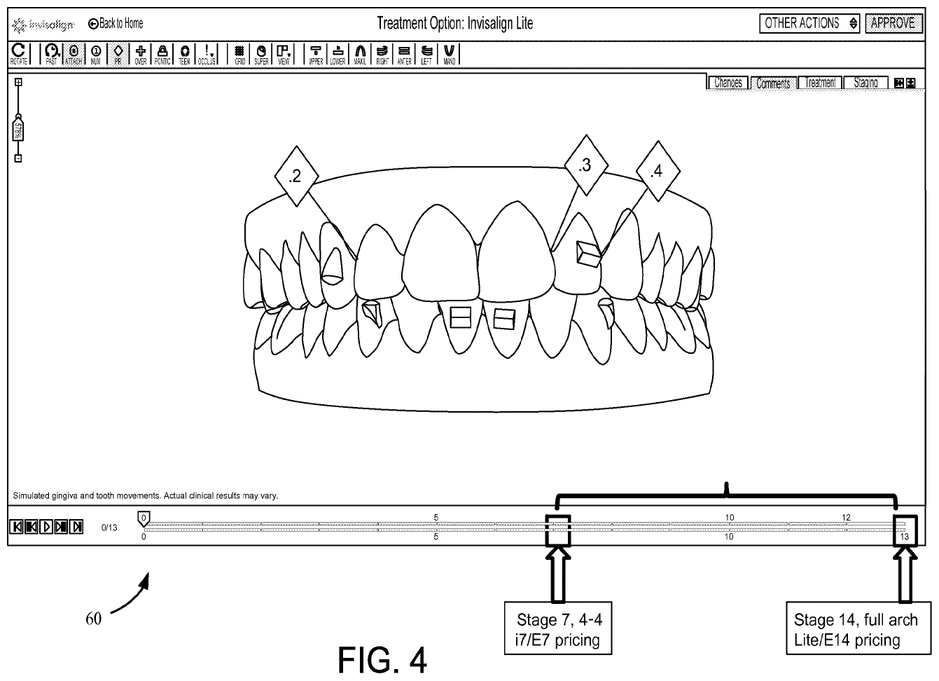

In another embodiment, the planning tool allows the user to select a treatment plan based the number of stages in a treatment plan where the tooth movement routes may differ from plan to plan. In this embodiment, the different treatment plans are also generated from a single initial data set. For example, FIG. 4 shows a screen shot 60 of a comparative treatment planning tool illustrating a graphical representation of a treatment stage in a treatment plan according to another embodiment. In this embodiment of the treatment planning tool, different treatment plans having different tooth movement routes or paths are provided and compared so that the treatment provider and/or patient can choose from different treatment plans/approaches based on the individual's needs. In the illustrated example, the scroll bar at the bottom of the screen shot 60 shows two different stages from two different treatment plans that can be compared: “Stage 7, 4-4, i7/E7 pricing” and “Stage 14, full arch, Lite/E14 pricing.” For example, in one treatment plan, there can be posterior movement in addition to anterior movement whereas the movement in another treatment plan is limited to only anterior movement. In the screenshot 60 in FIG. 4, Stage 7 and Stage 14 of the same treatment plan can be selected to see the predicted tooth positioning at these stages of the treatment plan. At the same time, the user can also see the respective pricing if these stages are selected to be the final stage of the treatment plan. In this example, the pricing at Stage 14 of the “4-4” treatment plan is more than if the same treatment is stopped at Stage 7. It will be understood that the “4-4” treatment plan is only an example. The treatment plan could be for any subgroup of teeth and any number of stages, and any combination thereof. For example, the treatment plan could be for “3-3” (anterior teeth only) or “5-5” or “7-7.” It will also be understood that the pricing terms (i.e., “i7/E7 pricing” and “Lite/E14 pricing”) used in the illustrated example of FIG. 4 are also only examples and are provided to illustrate that the comparative treatment planning tool can also provide relative pricing for different stages on the screen.

The screenshot 60 of FIG. 4 also shows Stage 14 of a treatment plan for the full arch (i.e., both anterior and posterior movement) and its pricing. The comparative treatment planning tool provides comparison of the different treatment stages of different treatment plans by allowing toggling between the treatment stages or superimposition of different stages over one another, as noted above. Although only Stage 7 and Stage 14 are shown in the illustrated screenshot 60, it will be understood that the user can select the number of stages for a treatment plan. Alternatively, the user can specify the desired pricing range and the treatment planning tool can provide the treatment plan options in which the number of stages and/or tooth movement routes can be selected for the desired pricing range.

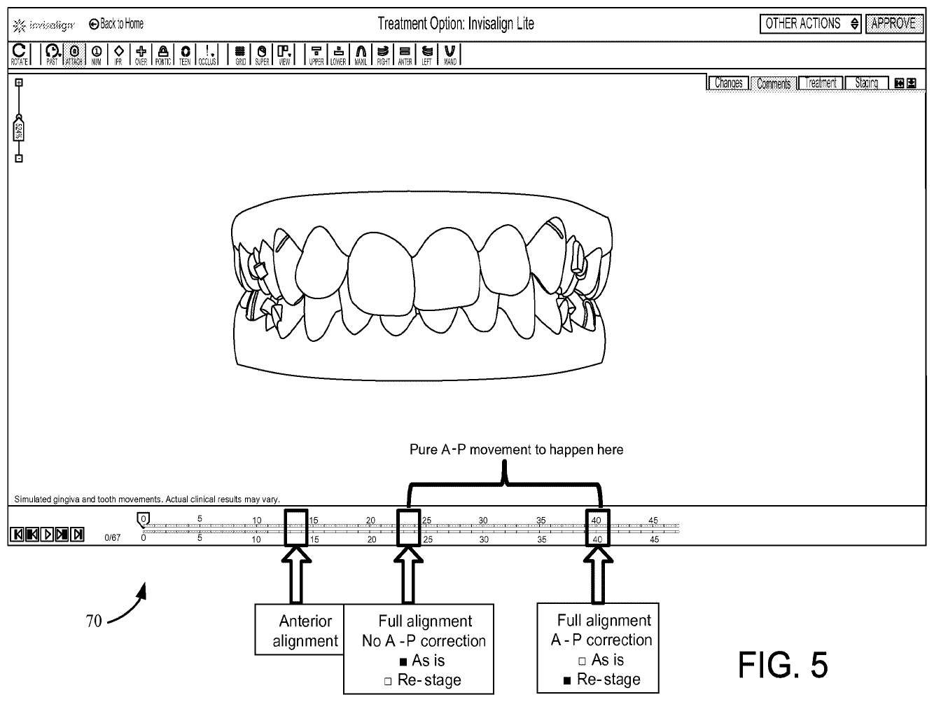

FIG. 5 shows a screen shot 70 of a comparative treatment planning tool illustrating a graphical representation of a treatment stage in a treatment plan according to yet another embodiment. In this embodiment, the comparative treatment planning tool provides different treatment plans from a single initial data set based on a treatment goal provided by the user. For example, a treatment goal of anterior alignment only can be provided. Alternatively, a treatment goal of full alignment can be provided. In some cases, a patient might choose to align only the teeth in the top arch as opposed to both the top and bottom arches. The user can use the treatment planning tool to compare the tooth positioning at various stages. Once the treatment provider determines which accept point to select, based on the complexity of treatment goal the patient wants to achieve, there would be different options, as described below.

One option is to manufacture aligners as is, with consecutive treatment goals being achieved (e.g., treatment would first achieve anterior alignment, then full alignment, then bite relationship correction, not having simultaneous movements, i.e., correcting anterior alignment while correcting class II). Another option is to re-stage the treatment with the final accept point as the sole treatment goal, thereby potentially skipping and/or consolidating previous accept point and therefore shortening treatment time. In the example provided, a re-staged treatment plan might achieve anterior and posterior alignment at the same time instead of consecutively. Or, a re-staged plan might begin with anterior alignment, then posterior alignment, and finish with more anterior alignment. The re-staging can be done to most efficiently move the teeth to reach the end goal. Different re-staged treatment plans (having different routes and/or alignment options) could be provided, depending on the patient's goals. If the patient has a date by which he or she would like anterior alignment (e.g., for a wedding), then, in the provided example, the treatment may not need to be re-staged and the anterior alignment would take place first. Yet another option is to provide restorative treatment at certain stages in addition to orthodontic treatment versus orthodontic treatment only throughout the treatment plan. It will be understood that the comparative treatment planning tool can be used to provide other treatment plans to allow a clinician to compare the different treatment plans and select one based on treatment duration, complexity, pricing, etc.

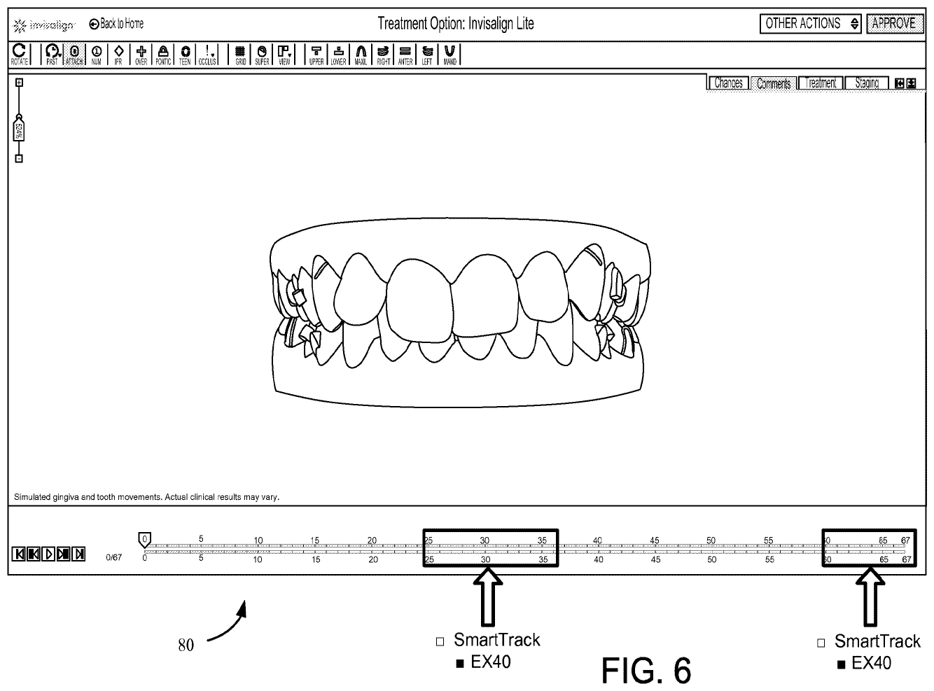

FIG. 6 shows a screen shot 80 of a comparative treatment planning tool illustrating a graphical representation of a treatment stage in a treatment plan according to another embodiment. According to the example shown in FIG. 6, the user can select different materials for aligners to achieve different types as well as different rates of movements. The treatment planning tool can provide the user with multiple accept points for batch shipping based on the material selected. For example, the aligners can be formed of a first material in Stages 1-14, a different material for Stages 15-20, and yet another material for Stages 21-24. In another example, the user can also select different features and attachments. Features and/or attachments can be used to change the shape of an aligner to achieve different types and/or rates of tooth movements. Attachments can include different geometric shapes, such as ovoid, tear-drop, ramp, hexagonal, and/or rectangular, and other shapes. The attachment elements can, for example, provide friction between the aligner and the teeth to increase retention of the aligner. That is, the attachment elements can create greater retention of the aligner on the teeth in a particular direction.

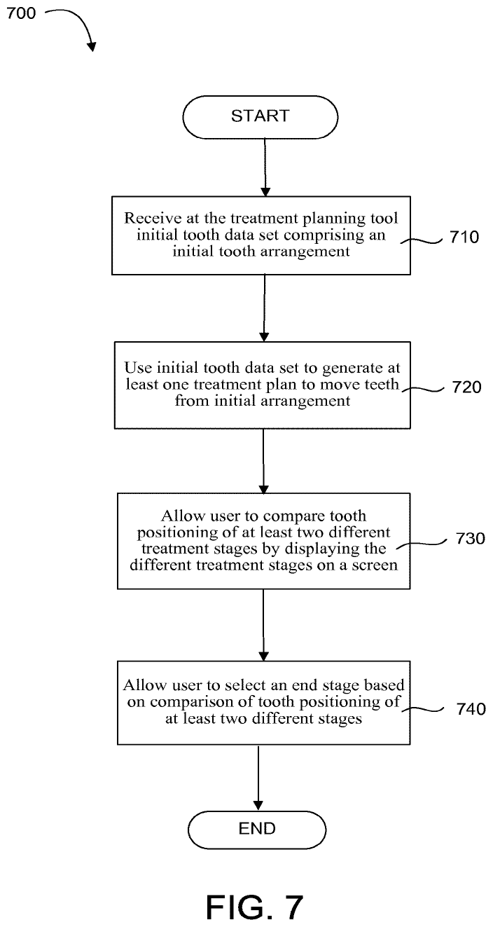

A method 700 of orthodontic treatment planning with a comparative orthodontic treatment planning tool will be described with reference to FIG. 7 and steps 710-740. In step 710, an initial tooth data set comprising an initial tooth arrangement is received at the treatment planning tool. In step 720, the initial tooth data set is used to generate at least one treatment plan to move teeth from the initial arrangement. Each treatment plan includes a plurality of treatment stages. A user is then allowed to compare the tooth positioning of at least two different treatment stages by displaying the at least two different treatment stages on a screen in step 730. The different treatment stages can be displayed either simultaneously (e.g., split screen display or superimposition of the different stages) or consecutively (e.g., toggling between displays of the different stages). In step 740, the user is allowed to select an end stage based on a comparison of tooth positioning of at least two different stages.

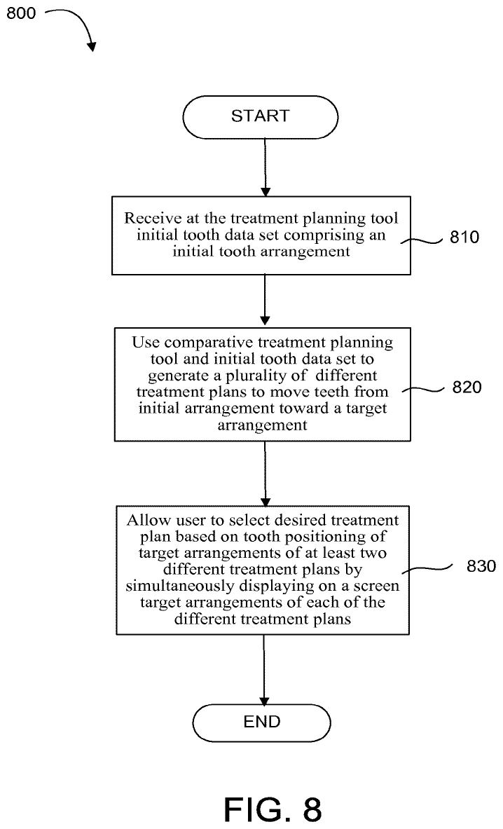

A method 800 of orthodontic treatment planning using an orthodontic treatment planning system comprising a computer and a comparative treatment planning tool will be described with reference to FIG. 8 and steps 810-830. In step 810, an initial tooth data set comprising an initial tooth arrangement is received at the computer. In step 820, the comparative treatment planning tool and the initial tooth data set are used to generate a plurality of different treatment plans to move teeth from the initial arrangement toward a target arrangement. Each of the treatment plans includes a plurality of treatment stages. In step 830, a user is allowed to select a desired treatment plan based on tooth positioning of target arrangements of at least two different treatment plans by simultaneously displaying on a screen the target arrangements of each of at least two different treatment plans.

The embodiments of the comparative treatment planning tool described herein allow patients and/or orthodontists to compare different treatment plans to determine the best treatment plan for the patient, based on different factors, such as time, cost, complexity, materials, etc. According to some embodiments, the treatment planner, such as a dentist or an orthodontist, receives a single input or set up for a particular patient. The set up includes the tooth data of the initial arrangement of the patient's teeth. Based on this single set up, the treatment planner and/or the patient can see and compare and then choose from different treatment options. The decision making can be based on one or more of the factors described herein.

In some embodiments, the treatment provider and/or patient can choose from different treatment plans based on the number of aligners desired. For example, if a patient desires a relatively short treatment duration, the treatment provider may choose a treatment plan that has relatively few aligners but provides significant tooth alignment (e.g., anterior alignment) to still achieve a noticeable and acceptable result. The incremental improvement of the appearance of the teeth that can be achieved with additional treatment stages (and aligners) may not be worth the added time (and expense) for some patients. Some patients, on the other hand, may want to take the time (and spend the money) to achieve the best outcome possible. The treatment provider and/or patient can also choose from different materials for aligners as well as different features, attachments, and shapes for the aligners. In some cases, the patient may want a treatment plan having a short duration to improve the appearance of his or her teeth for an event (e.g., wedding) and then continue treatment later.

Although only a few embodiments of the invention have been described in detail, it should be appreciated that the invention may be implemented in many other forms without departing from the spirit or scope of the invention. In view of all of the foregoing, it should be apparent that the present embodiments are illustrative and not restrictive and the invention is not limited to the details given herein, but may be modified within the scope and equivalents of the appended claims.

1. (canceled)

2. A method for planning an orthodontic treatment, the method comprising:
transmitting an initial tooth data set to an orthodontic treatment planning tool, the initial tooth data set comprising an initial arrangement of a patient's teeth;
receiving a plurality of treatment plans for the patient's teeth from the orthodontic treatment planning tool, wherein each treatment plan comprises a plurality of treatment stages to move the patient's teeth from the initial arrangement toward a respective final arrangement, and wherein the plurality of treatment plans differ from each other based on one or more treatment factors;
receiving, through a display, a comparison of a treatment stage of a first treatment plan of the plurality of treatment plans to a treatment stage of a second treatment plan of the plurality of treatment plans; and
providing a user input selecting one of the first treatment plan or the second treatment plan based on the comparison.

3. The method of claim 2, further comprising:
receiving, through the display, one or more additional options for customizing the plurality of treatment plans, and
providing a user input selecting a subset of the one or more additional options for viewing.

4. The method of claim 3, wherein the comparison of the treatment stage of the first treatment plan and the treatment stage of the second treatment plan is received in response to the user input selecting the subset of the one or more additional options for viewing.

5. The method of claim 3, wherein the one or more additional options are based on one or more of the following: treatment cost, treatment duration, number of aligners, material selection, or attachment selection.

6. The method of claim 2, further comprising administering, to the patient, one or more orthodontic appliances corresponding to one or more treatment stages of the selected one of the first treatment plan or the second treatment plan.

7. The method of claim 2, wherein the first and second treatment plans have different numbers of treatment stages.

8. The method of claim 2, wherein the first and second treatment plans have different final arrangements.

9. The method of claim 2, wherein the first and second treatment plans have different tooth movement routes.

10. The method of claim 2, wherein the first and second treatment plans move different subgroups of the patient's teeth.

11. The method of claim 2, wherein the first and second treatment plans use different attachments to move the patient's teeth.

12. The method of claim 2, wherein the first and second treatment plans differ from each other with respect to one or more of the following: use of interproximal reduction, use of extraction, or use of restorative treatment.

13. The method of claim 2, wherein the one or more treatment factors comprise one or more of treatment cost, treatment duration, number of aligners, material selection, or attachment selection.

14. The method of claim 2, wherein the plurality of treatment plans each comply with one or more constraints presented in the one or more treatment factors.

15. A method for planning a dental treatment, the method comprising:
transmitting a clinical prescription for treating a patient's teeth to a dental treatment planning tool;
receiving a plurality of treatment plans for the patient's teeth from the dental treatment planning tool, wherein each treatment plan comprises a plurality of treatment stages to move the patient's teeth from an initial arrangement toward a respective final arrangement, and wherein the plurality of treatment plans differ from each other based on one or more treatment factors;
receiving, through a display, a treatment stage of a first treatment plan of the plurality of treatment plans and a treatment stage of a second treatment plan of the plurality of treatment plans for comparison; and
providing a user input selecting one of the first treatment plan or the second treatment plan based on the comparison.

16. The method of claim 15, further comprising:
receiving, through the display, one or more additional options for customizing the plurality of treatment plans, and
providing a user input selecting a subset of the one or more additional options for viewing.

17. The method of claim 16, wherein the treatment stage of the first treatment plan and the treatment stage of the second treatment plan are received in response to the user input selecting the subset of the one or more additional options for viewing.

18. The method of claim 16, wherein the one or more additional options are based on one or more of the following: treatment cost, treatment duration, number of aligners, material selection, or attachment selection.

19. The method of claim 15, further comprising administering, to the patient, one or more dental appliances corresponding to one or more treatment stages of the selected one of the first treatment plan or the second treatment plan.

20. The method of claim 15, wherein the first and second treatment plans have different numbers of treatment stages.

21. The method of claim 15, wherein the first and second treatment plans have different final arrangements.

22. The method of claim 15, wherein the first and second treatment plans have different tooth movement routes.

23. The method of claim 15, wherein the first and second treatment plans move different subgroups of the patient's teeth.

24. The method of claim 15, wherein the first and second treatment plans use different attachments to move the patient's teeth.

25. The method of claim 15, wherein the first and second treatment plans differ from each other with respect to one or more of the following: use of interproximal reduction, use of extraction, or use of restorative treatment.

26. The method of claim 15, wherein the one or more treatment factors comprise one or more of treatment cost, treatment duration, number of aligners, material selection, or attachment selection.

27. The method of claim 15, wherein the plurality of treatment plans each comply with one or more constraints presented in the one or more treatment factors.
